(19) United States
(12) Patent Application Publication
(10) Pub. No.: US 2024/0181222 A1
(43) Pub. Date: Jun. 6, 2024

(54) SAFETY CANNULA ASSEMBLY

(71) Applicant: SARSTEDT AG & CO. KG, Nuembrecht (DE)

(72) Inventor: Viktor HAUPT, Nuembrecht (DE)

(21) Appl. No.: 18/537,296

(22) Filed: Dec. 12, 2023

Related U.S. Application Data
(63) Continuation of application No. PCT/EP22/65995, filed on Aug. 13, 2022.

(30) Foreign Application Priority Data
Jun. 16, 2021 (DE) DE 102021115556.8

(51) Int. Cl. A61M 25/06; A61M 39/02

(52) U.S. Cl. CPC A61M 25/0631; A61M 25/0618; A61M 39/0247; A61M 2039/0202; A61M 2039/0205; A61M 2039/0258

(57) ABSTRACT

A safety cannula assembly, comprising: a cannula to puncture human or animal tissue, wherein a distal end section of the cannula is provided with a tip; a sliding body including a distal end section including the cannula and a proximal end section including a flexible tube, wherein a flow connection between the tip and a proximal end of the flexible tube runs through the sliding body; a main body in which the sliding body is displaceable from an operating position where the tip of the cannula is arranged outside of the main body into a safety position where the tip of the cannula is arranged inside the main body, wherein the main body is assembled from at least three main body parts, including a first main body part configured sleeve shaped and enveloping a distal end section of the sliding body in the operating position of the sliding body.

### RELATED APPLICATIONS

This application is a continuation of International patent application PCT/EP2022/065995 filed on Jun. 13, 2022 claiming priority from German Patent application DE 10 2021 115 556.8 filed on Jun. 16, 2021, both of which are incorporated in their entirely by this reference.

### FIELD OF THE INVENTION

The invention relates to a safety cannula assembly,

The positional or directional indication “proximal” according to the invention indicates an arrangement oriented towards a body of a person using the safety cannula assembly, in particular an arrangement or position oriented towards a hand of the person holding the safety cannula arrangement, whereas “distal” indicates an arrangement oriented away from the body or the hand. Consequently a proximal end of an object, thus also of the safety cannula arrangement according to the invention is closer to the body of the person than a distal end.

Cannula arrangements according to the invention are typically used in medical applications to draw a bodily fluid, in particular venous blood from a patient or to feed a fluid into a fluid vessel of the human body. Safety cannula assemblies are used to prevent injuries from needle punctures after use in an optimum manner and to provide maximum comfort for the person using the cannula assembly and to prevent pain and trauma for the patient using the cannula arrangement including activating the needle protection. A pull back movement of the needle after completion of the blood draw or infusion is performed automatically for a passive needle protection without further interaction by the user, whereas an active needle protection requires an intentional action by the person using the cannula arrangement to move the cannula into the safety position. The instant invention primarily relates to safety cannula assemblies with active needle protection.

The main body according to the invention is defined as performing a housing function and having an enveloped interior space that includes at least a portion of the sliding body in the operating position, wherein at least the cannula itself including its tip is housed in the interior space of the sliding body in the safety position, this means after displacing the sliding body, in order to prevent puncture wounds from the tip and also contact with the needle where contaminated or infectious body substance might be arranged. The main body may have a single part configuration or a multi-part configuration according to the invention wherein the individual main body parts can be inserted or clipped into one another or connected with one another in case of the multi-part configuration. Also, the connection of plural main body parts with so called film hinges is conceivable in the context of the instant invention. Walls of the main body do not have to be completely closed in this context but can also have cut outs, interruptions, slots, or other perforations so that a hermetic sealing of the interior towards the ambient does not have to be provided.

An actuation element according to the instant invention is an element configured to impart a required force upon a sliding body functioning as a drive to move the sliding body from the operating position into the safety position. A drive element and an associated energy storage device can be separate from each other. However, the two functions can be combined in one element, in particular configured as spring element that either stores mechanical energy in the form of a coil spring and/or pressure energy like e.g. in a preloaded gas storage device.

A sliding body according to this application is an element which receives the cannula at a distal end and which is connected with a tube at a proximal end. The sliding body is typically also designated as cannula holder or needle carrier.

### BACKGROUND OF THE INVENTION

EP 2 108 394 B1 discloses a protective cover arrangement for a cannula where the main body is assembled from three main body parts. A first main body part is formed by a distal sleeve shaped end section where the cannula is arranged in the retracted, this means safety position of the sliding body so that injuries by needle puncture are avoided. The second main body part is integrally connected in a rigid manner, this means without link or hinge with the first sleeve shaped main body part and shaped as a semi-cylindrical groove that expands at the proximal end into a trapezoid shape. The second main body part is also groove or tub shaped in the expanded section, however with an expanded width. A proximal face wall has a semi-circular section. The known arrangement, however, includes a third main body part that is connected with the second main body part by a film hinge running parallel to the longitudinal axis of the sliding body. The film hinge is arranged in the broadened proximal end section of the second main body part. The third main body part has a groove or tub shape that is complementary to the end section of the second main body part. The second and the third main body part are arranged adjacent and complementary to one another in the expanded width end section of the second main body part and are fixed at one another by a snap hook connection. The third main body part includes a semi-circular recess in its proximal face wall wherein the semi-circular recess forms a circular pass-through opening for the flexible hose and the sliding body together with the semi-circular recess in the proximal face wall of the second main body part. It is a disadvantage of this known safety cannula arrangement that the main body is open in a center portion between the sleeve shaped first main body part and the distal face of the second main body part since the main body is only made from the half shell of the second main body part at this location. This runs the risk that a contamination of persons occurs by touching the freely accessible cannula outside of the tip portion of the cannula that is wetted with a bodily fluid after using the device and transferring the needle carrier into the safety position. Additionally the known safety cannula arrangement does not include an actuation element that facilitates transferring the sliding body self-acting from the operating position into the safety position by manual triggering. A very similar safety cannular arrangement is also known from WO 2013/068855A1 by the same applicant.

EP 2 509 674 B1 describes a main body of a safety cannula arrangement where two main body half shells are connected by a film hinge that runs transversal to a longitudinal axis of the main body and that leaves a pass-through opening for the cannula between two hinge sections. A respective longitudinally extending slot is arranged in opposite narrow sides of the main body that are arranged opposite to each other when the two main body parts are joined, wherein a grip lobe respectively connected with the needle carrier passes through the slot and is movable in the axial direction together with the needle carrier. An actuation element for a self-acting transfer of the sliding body from the operating position into the safety position is not provided in this known safety needle arrangement.

Additionally WO 2011/10 0039A1 discloses a safety cannula arrangement where no tube is connected to the rear end of the sliding body or the cannula carrier but where the needle protrudes on both sides of the sliding body and the rear free end of the needle is configured to directly receive the blood drawing vial. Therefore, the sliding body according to WO 20 11/10 00 39 A1 is configured with a sufficiently large diameter to receive the blood drawing vial and open at the proximal end to facilitate the insertion.

The safety function of this known cannula arrangement is provided in that a receiving body for the sliding body and the double cannula supported therein is moved in a direction transversal to the longitudinal axis of the cannula into the space for the blood drawing vial that is not present any more after completing the blood draw and decoupling and pulling the blood drawing vial out of the main body so that a trigger member releases a retraction movement of the cannula including the sliding body when a receiving cross section of the sliding body is pushed over the proximal free cannula end and thus facilitates a pull-back movement of the sliding body including the cannula in an interior of the receiving body. The receiving body and the sliding body are thus in an arrangement where they are inclined or kinked relative to each other which, however, is not a problem for the movement of the sliding body on a curved path due to the sufficiently small angle between the two longitudinal axes.

The trigger member and the locking principle implemented by the trigger member include a key hole that facilitates a locking of the sliding body in the operating position through a narrow cross section of the key hole but facilitates a passage of the sliding body with an enlarged cross section in the trigger position. Though WO 2011/10 0039 includes a film hinge for connecting two main body parts the known configuration is not suitable to implement a plurality of main body parts and their connection by a respective film hinge.

U.S. Pat. No. 5,746,215 A discloses a safety cannula arrangement where the cannula has to be pulled out into an operating position from an initially entered safety position by an externally accessible slide. During the transfer out of the main body a spring element is lengthened and thus preloaded wherein the spring element due to pressure upon a trigger member contracts again after completing the blood draw or the puncture so that the sliding body including the cannula is returned into the safety position.

EP 2 985 049 A1 discloses a butterfly needle protective sleeve that includes a main body, configured to receive a butterfly needle wherein the main body is configured as an integral one-piece injection molded component. The butterfly needle includes a needle section, a lobe section that protrudes from the main body and a grip section attached at the lobe section. Thus, an end of the grip section is connected with the needle section and the other end is configured to be connected with the tube. The butterfly needle is displaceable between an operating condition with a protruding needle tip and a safety condition without protruding needle tip. According to EP 2 985 049 A1 the main body includes a needle tip protective head which includes an upper cover and a lower cover. The needle tip protective head is configured as a cylinder including an axially open needle hole. The upper cover and the lower cover are formed by elongated components that extend in an axial direction of the main body and respectively include a distal end and a proximal end. Thus, the distal ends of the upper cover and of the lower cover are connected by film hinges with the cylindrical protective head of the main body.

Last not least EP 1 448 260 A1 discloses a cover housing of a lobed cannula where the housing or main body is assembled from two complementary half shells that are connected by a film hinge. The two half shells are connected by a film hinge running parallel to a longitudinal axis of the cover housing or the needle carrier. Snap hooks configured to fix both housing halves in the closed condition are arranged on a longitudinal side of the housing arranged opposite to the film hinge. This known housing configuration includes slots on opposite longitudinal sides wherein the slots run in the longitudinal direction and facilitate a displacement of the sliding body including the retaining lobes arranged thereon and protruding in opposite directions.

### BRIEF SUMMARY OF THE INVENTION

Thus, it is an object of the invention to provide a safety cannula arrangement where the assembly process is simplified and the positioning of the actuation element and the generation of its preload is made easier while reducing manufacturing complexity.

Improving upon the generic safety cannula arrangement recited supra the object is achieved by A safety cannula assembly, comprising: a cannula configured to puncture human or animal tissue, wherein a distal end section of the cannula is provided with a tip; a sliding body including a distal end section including the cannula and a proximal end section including a flexible tube, wherein a flow connection between the tip of the cannula and a proximal end of the flexible tube runs through the sliding body; a main body in which the sliding body is displaceable from an operating position where the tip of the cannula is arranged outside of the main body into a safety position where the tip of the cannula is arranged inside the main body, wherein the main body is assembled from at least three main body parts, including a first main body part configured sleeve shaped and enveloping a distal end section of the sliding body in the operating position of the sliding body, a second main body part adjoining the first main body part in the proximal direction and connected with the first main body part, wherein the second main body part has a U-shaped, L-shaped, C-shaped, or lid-shaped cross section, a third main body part joinable with the second main body part to form a hollow body that is open at two opposite faces, wherein the third main body part is configured lid-shaped, C-shaped, L-shaped, or U-shaped and connected by a film hinge with the second main body part or the first main body part; an actuation element arranged between the main body and the sliding body and configured to displace the sliding body from the operating position to the safety position; and a trigger mechanism arranged at the main body and configured to trigger a displacement of the sliding body from the operating position to the safety position, wherein the trigger mechanism includes at least one trigger member loadable with a radially oriented compressive force or a torque by a person using the safety cannula assembly, and at least one locking element displaceable by loading with the pressure force or the torque from a locking position where the locking element engages the sliding body and locks the sliding body in the operating position to a trigger position, where the locking element does not engage the sliding body so that the sliding body moves into the safety position, wherein the actuation element is supported at a support surface of the first main body part with a distal end of the actuation element.

The cannula arrangement according to the invention which is primarily used as a blood drawing device has many advantages during assembly, in particular during insertion of the sliding body into the main body, thus initially the first main body part and the adjoining second main body part are provided, both of which are arranged axially behind one another in the direction of the longitudinal axis of the main body or the sliding body or brought into linear alignment with each other in case of a film hinge connection between the first and the second main body part. Then, the sliding body and the actuation element are joined and inserted as a unit into the first and the second main body part or the actuation element is initially inserted without preload into the first main body part and the second main body part and the sliding body is added only in a next step and the actuation element is placed into the preloaded condition by the sliding element.

The sleeve shaped first main body part has a substantial advantage with respect to a safe precisely fitting positioning of the actuation element in the main body part. In particular when a coil spring is used as an actuation element the coil spring can be aligned coaxial to the sleeve shaped first main body part and inserted so that the coil spring is then reliably fixed at its distal end against sliding or escaping or skipping away while transitioning into the preloaded condition.

The third main body part is then connected with the second main body part as a last step in the assembly sequence so that the main body is completed and provided in its final form.

In order to simplify handling or to provide an alternative handling in addition to gripping the housing part of the main body it is proposed to provide the main body with a respective gripping lobe at two opposite sides. The gripping lobes can be made from a rubber elastic material and can be pressed against one another in a known manner by gripping them with two fingers in a portion above the main body until they are in contact with each other. An embodiment with two gripping lobes increases comfort for the user. An application with only gripping lobe or no gripping lobe at all is also conceivable.

An advantageous embodiment of the invention provides that the actuation element is supported with its distal end at a shoulder formed in an interior of the first main body part or at a face wall forming a distal termination of the first main body part. This assures particularly simple and reliable fixing of the actuation element while transferring the actuation element into the preloaded condition.

In another advantageous embodiment of the arrangement according to the invention the gripping lobes can be connected to a sleeve shaped connector that is slid onto the main body. Thus, an overlapping portion between the connector and the main body envelops at least an axial section of the first main body part as well as a respective axial section of the second main body part and of the third main body part. The sleeve shaped connector thus not only causes a safe and firm connection of the gripping lobes at the main body but stabilizes the main body forming a jacket or a clamp ring which provides an additional safety in the assembled condition for the assembly of the main body from several main body parts.

A first embodiment of the first and second main body part, is characterized in that both parts are rigidly connected with one another. Thus, there is no pivotability between the two main body parts, in particular there is no film hinge between the two main body parts. In this embodiment the alignment of the first body part with the second main body part is always automatically provided and a degree of freedom is eliminated during the assembly, wherein this degree of freedom would otherwise need to be eliminated by respective fixing and guiding the components.

As an alternative the embodiment recited supra, it is also possible to connect the first main body part and the second main body part with one another by a film hinge. In this case a hinge axis of the film hinge should be aligned perpendicular to and at a distance from the longitudinal axis of the sliding body.

With respect to the third main body part two basic cases of connection with the remaining main body parts are feasible. In a first case the third main body part and the second main body part can be connected with one another by a film hinge. The hinge axis of the film hinge can thus be aligned parallel or perpendicular to the longitudinal axis of the sliding body. In this variant the second main body part forms a center element of a three-element chain formed by the three main body parts which are connected by two film hinges, wherein the second main body part is connected with both film hinges.

As an alternative to the embodiment recited supra there is an option to connect the third main body part and the second main body part with one another by a film hinge. In this case like in the case where the first and the second main body part are connected, the hinge axis of the film hinge is aligned perpendicular to and at a distance from the longitudinal axis of the sliding body. In the latter case a symmetrical arrangement can be provided where the first main body part forms the center link of the three-element chain and the two end-links of the chain are formed by advantageously identically configured main body parts. In this constellation, the second and the third main body parts are advantageously synchronously pivoted about the respective film hinge axes onto the longitudinal axis of the sliding body previously inserted into the first main body part and brought in contact with one another and fixed relative to one another.

For reasons of symmetry and in order to simplify assembly the two film hinges should have the same distance from the tip of the cannula viewed in the axial direction in the latter case. By the same token the two film hinges should have the same distance respectively from the longitudinal axis of the sliding body.

In an advantageous embodiment of the invention, the first main body part
is rotation symmetrical in a distal section, and/or
cuboid in a proximal section,
wherein a respective film hinge is advantageously arranged at opposite proximal edges of the cuboid proximal section. This embodiment facilitates a reliable arrangement of the distal end of the actuation element or sliding body in the rotation symmetrical distal section and a rotation safety of the sliding body in the cuboid proximal section if the sliding body has a cross sectional shape which prevents a rotation within the proximal cuboid section that advantageously has a rectangular cross section, in particular a square cross section.

According to an advantageous embodiment of the safety cannula arrangement according to the invention, the rotation symmetrical section of the first main body part has a cylindrical tip section and also has a cylindrical transition section configured to receive the actuation element that is compressed in the operating position, wherein the removable tubular cannula protection is advantageously slid onto the tip section.

According to an advantageous embodiment, the second main body part and the third main body part, in joined condition
form a gripping portion adjoining the film hinges in the proximal direction wherein the gripping portion is advantageously configured with ribs or a plurality of protrusions on an outer enveloping surface in order to improve gripping, and/or
form a trigger portion, adjoining the gripping portion in the proximal direction, wherein the trigger portion includes a trigger mechanism and/or
form a closure portion arranged at a proximal end of the main body wherein the second main body part and the third main body part are connected with one another by positive form locking through snap hooks and/or interlocking lugs.

According to an advantageous embodiment of the invention, the first main body part forms a distal end of the main body.

### BRIEF DESCRIPTION OF THE DRAWINGS

The invention is subsequently described based on an embodiment with reference to drawing figures, wherein

FIGS. 1 and 2: illustrate a safety cannula arrangement in two perspective views including a cannula protection plugged on distally;

FIG. 3: illustrates an exploded view of the safety cannula arrangement;

FIG. 4: illustrates a perspective view of the main body including the cannula protruding there from;

FIG. 5: is similar to FIG. 4 but shows a section of the main body;

FIG. 6: illustrates a released first main body part including a sliding body inserted therein;

FIG. 7: illustrates the sliding body with a cannula, actuation element and a section of a flexible tube;

FIG. 8: is similar to FIG. 7, but does not include the actuation element;

FIG. 9: illustrates the perspective view of the main body including a second and third main body part folded out laterally;

FIG. 10: illustrates a perspective view of the first and second main body part;

FIG. 10A: is similar to FIG. 10, but shows a different perspective;

FIGS. 11 and 12: illustrates a longitudinal sectional view according to FIGS. 1 and 2 showing the sliding body in the operating position;

FIG. 12A: illustrates a blown-up detail of FIG. 12

FIGS. 13 through 16: illustrate a sectional view along the line I-I through the safety cannula arrangement according to FIGS. 1 and 2 in different positions of the trigger members and locking elements;

FIG. 17: illustrates a perspective view of the safety needle in a sectional view along the line Il-11 in FIGS. 1 and 2;

FIG. 18: illustrates a trigger mechanism in a sectional view along the line Ill-Ill like in FIG. 1 looking in the proximal direction;

FIG. 19: illustrates a longitudinal sectional view through the safety needle arrangement according to FIGS. 1 and 2 in the safety position of the sliding body;

FIG. 19A: illustrates a blown-up detail of FIG. 19;

FIG. 20: Illustrates a sectional view of the safety cannula arrangement according to FIGS. 1 and 2 along the sectional like IV-IV with the sliding body in the safety position in the distal viewing direction;

FIG. 21: is similar to FIG. 18, however shows the safety position of the sliding body; and

FIG. 22: Illustrates a cross sectional view of the safety cannula arrangement according to FIGS. 1 and 2 along the line V-V in the distal viewing direction.

### DETAILED DESCRIPTION OF THE INVENTION

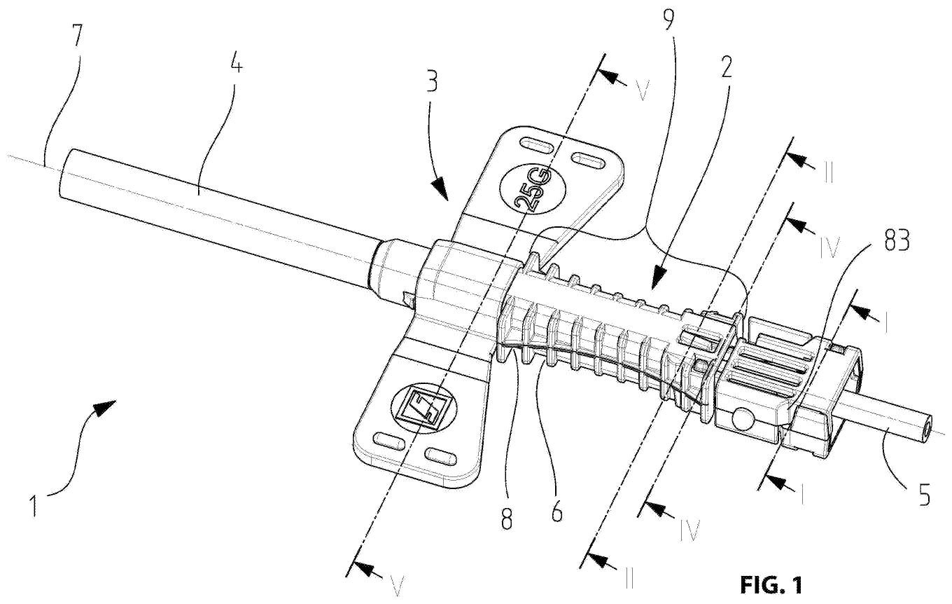

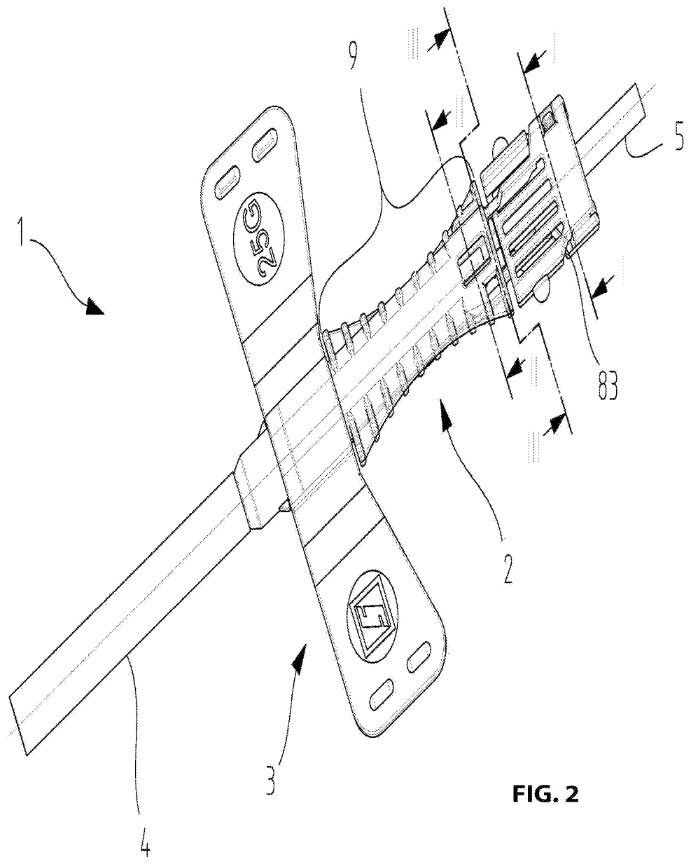

FIGS. 1 and 2 show a safety cannula assembly 1 in different perspectives in shipped configuration including a main body 2 to having an elongated housing, a lobe module 3 arranged at the main body, a tubular cannula protection 4 slid over a distally protruding cannula and a flexible tube 5 that is cut off for illustration purposes. The cut-off tube 5 that is connected at a proximal end section of a sliding body that is not illustrated in FIGS. 1 and 2 has a length of 5 cm to 30 cm and includes an adapter at a proximal end wherein the adapter establishes connectivity with additional handling devices used for blood drawing. The main body 2 includes a plurality of transversal ribs 6 running perpendicular to a longitudinal axis 7 of the main body or also of the entire safety cannula assembly including the sliding body, wherein a protrusion of the transversal ribs beyond the rounded cuboid base geometry of the grip portion 9 of the main body 2 initially decreases from a maximum protrusion at the transversal ribs 6 adjacent to the lobe module 3 towards the proximal end of the grip portion of the main body 2 and subsequently increases again. The transversal ribs 6 running equidistant and parallel to one another are connected with one another and stabilized at each side by a longitudinal rib 8 arranged in a symmetry plane of the main body 2. This way a particularly safe and ergonomic grip is facilitated for two fingers of a person using the safety cannula arrangement 1 wherein the two fingers grip the grip portion 9 of the main body from opposite sides which facilitates handling.

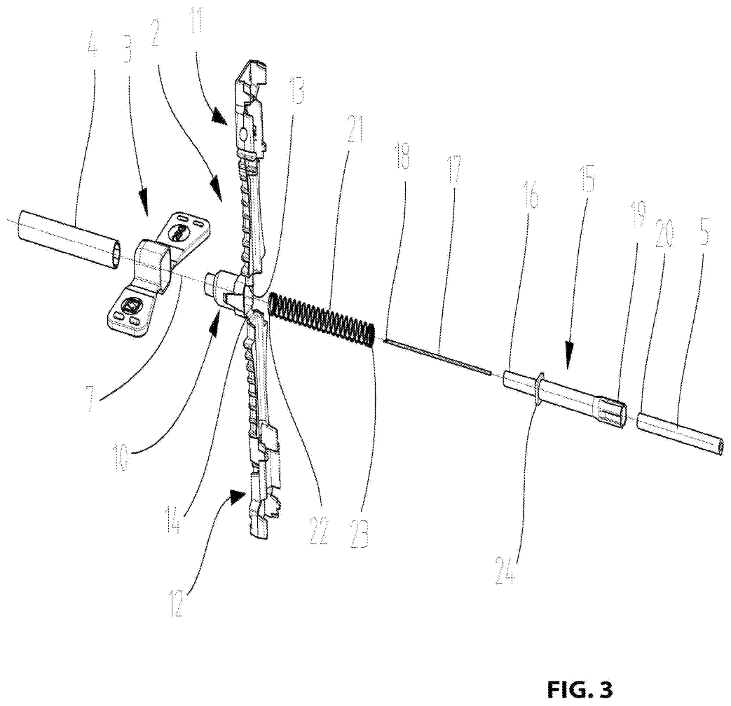

It is evident from the exploded view according to FIG. 3 that the main body 2 is assembled from three main body parts 10, 11 and 12. Thus, from a sleeve shaped first main body part 10 which extends the most in the distal direction and two half shell shaped main body parts including the second main body part 11 and the third main body part 12. The second main body part 11 and third 12 are respectively connected with the first main body part 10 by a film hinge 13, 14, and thus pivotally movable about a respective hinge axis of the film hinge 13, 14 relative to the first main body part 10. The two main body parts 11 and 12 are identically configured but arranged symmetrical relative to a symmetrical plane which is parallel to the two film hinges 13, 14 and runs through the longitudinal axis 7 of the main body.

The inner cavity of the main body 2 includes an essentially hollow cylindrical sliding body 15 that is movably supported in the axial direction, this means in the direction of the longitudinal axis 7 relative to the main body 2. A cannula 17 including a tip 18 formed at a distal end by a ground surface is inserted into the sliding body and sealed therein. A proximal end section 19 that is formed as a socket section is inserted with a distal end section 20 of the tube 5 which is sealed therein. Therefore, the sliding body 15, the canula 17, and the tube 5 form a firmly connected unit whose components besides the flexibility of the tube 5 are not movable relative to one another. An activation element 21 configured as a coil spring is externally slid onto the distal end section 16 of the sliding body 15 in order to support the sliding body 15 in the operating position under a preload in the main body 2 and in order to provide a propulsion for a pullback movement of the sliding body 15 relative to the main body 2 in the proximal direction, wherein the coil spring is supported with a distal end 22 in the first main body part 10 and supported with a proximal end 23 at a flange 24 that has a rectangular cross section and that protrudes beyond an outer enveloping surface of the sliding body 15.

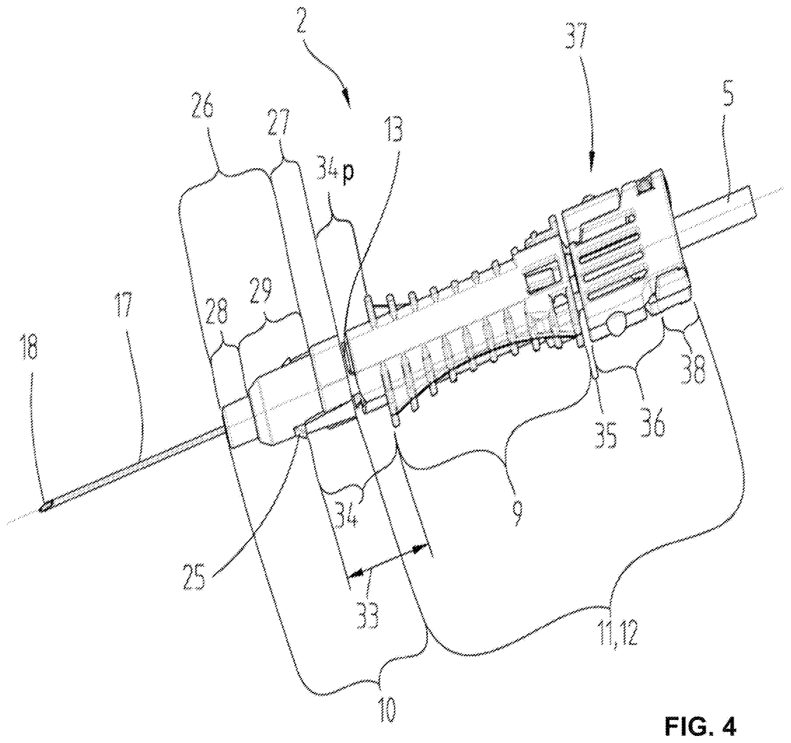

Subsequently the configuration of the main body 2 made from the main body parts 10-12 and the cooperation of the main body 2 with the sliding body 15 is described in more detail with reference to FIGS. 4 through 10.

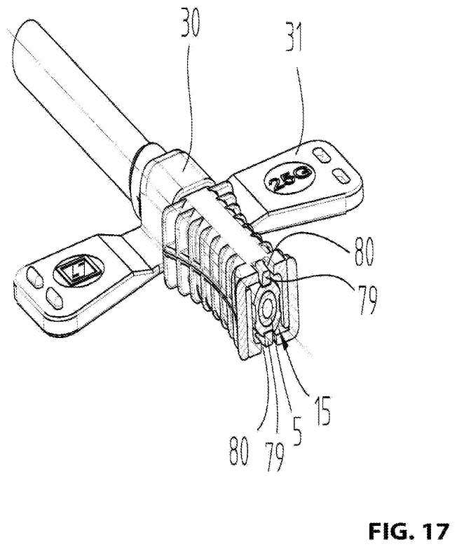

It is evident from FIG. 4 that the main body 2 can be divided into different portions in the axial direction. Starting at a distal end of the main body 2 the sleeve shaped first main body part 10 includes a rotation section that is rotation symmetrical besides two interlocking lugs 25 for fixing the lobe module 26 and an approximately cuboid section 27 adjacent in the proximal direction. The rotation symmetrical section 26 is divided into a cylindrical tip section 28 onto which the cannula protector 4 not illustrated in FIG. 4 but illustrated in FIGS. 1 and 2 can be slid and a cylindrical transition section 29 that has a conical taper at a distal end and that includes a distal end section 16 of the sliding body 15 including an actuation element 21 slid thereon. As evident from FIGS. 3, 1 and 2 the lobe module 3 is slid onto the main body 2 from a distal end of the main body with a center connection part 30 of the lobe module 3 where a respective gripping lobe 31 is integrally provided at opposite longitudinal sides of the lobe module 3 as evident from FIG. 17. An axially measured length 32 of the connection part 31 of the lobe module 3 thus corresponds to a length 33 of an overlapping portion 34 that extends over the first main body part 10 and also over the joined second main body part 11 and the third main body part 12 and that can be divided into the sections 34d and 34p wherein the first distal section 34d overlaps the first main body part 10 and the proximal section 34p overlaps the second and the third main body part 11, 12. The overlapping portion 34 includes an entirety of the cuboid section 27 in the portion of the first main body part 10 and includes a portion of the substantially cylindrical transition section 29, thus up to the interlocking lugs 25 included therein. The connection part 30 of the lobe module 3 thus bridges in particular also the portion of the two film hinges 13, 14 and a gap portion included therein between the first main body part 10 and the second main body part 11 and the third main body part 12. Starting from a proximal end of the overlapping portion 34 the grip portion 9 described supra adjoins in the proximal direction wherein the grip portion is jointly formed by the two main body parts 11 and 12. A circumferential notched portion 35 is arranged further in the proximal direction and a trigger portion 36 adjoins further in the proximal direction and includes a trigger mechanism 37 that will be described in more detail infra. A proximal end section of the main body 2 is formed by a closure portion 38 wherein the second main body part 11 and the third main body part 12 are connected with one another by snap hooks by positive form locking. An opening of the main body 2 provided by a pivot movement of the two main body parts 11 and 12 is thus prevented on the one hand side by the snap hooks in the closure portion 38 and on the other hand side by the connector 30 of the lobe module 3 that is slid over.

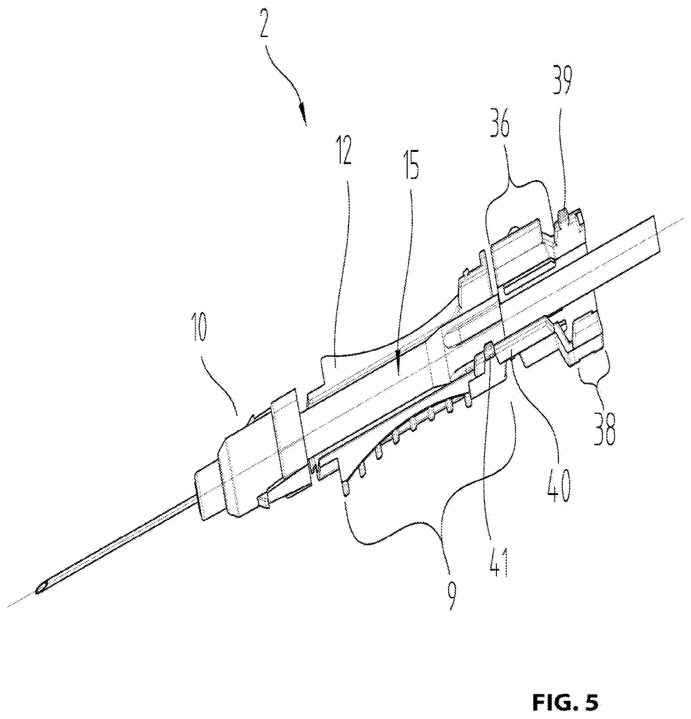

FIG. 5 shows how the sliding body 15 is arranged in the interior cavity of the main body 2 not showing the second main body part 11 but only the lower third main body part 12. The distal end section 16 of the sliding body 15 and the actuation element 21 are not shown because they are arranged covered in the sleeve shaped first main body part 10. The closure portion 38 shows a snap hook 39 that cooperates with an accordingly adapted recess in the second main body part 11 in a joined condition of the two main body parts 11 and 12 and that forms a closure that cannot be disengaged without destruction. The trigger portion 36 and the grip portion 9 are exclusively connected by a connection bar 40 in each of the two main body parts 11, 12 wherein the connection bar is connected with its proximal end with the grip portion 9 and connected with the trigger portion 36 at its distal end. It is appreciated that the entire second and third main body part 11, 12 and the main body 2 made from the three main body parts 10, 11, 12 including the film hinges 13, 14 is produced as one integral injection molded component. FIG. 5 shows an additional snap hook 41 at the main body part 12, wherein the snap hook 41 is arranged in the grip portion 9 and also cooperates with an accordingly adapted recess in the second main body part 11 and keeps the two main body parts 11 and 12 together in addition to the snap hooks 39 and the connector 30 of the lobe module 3.

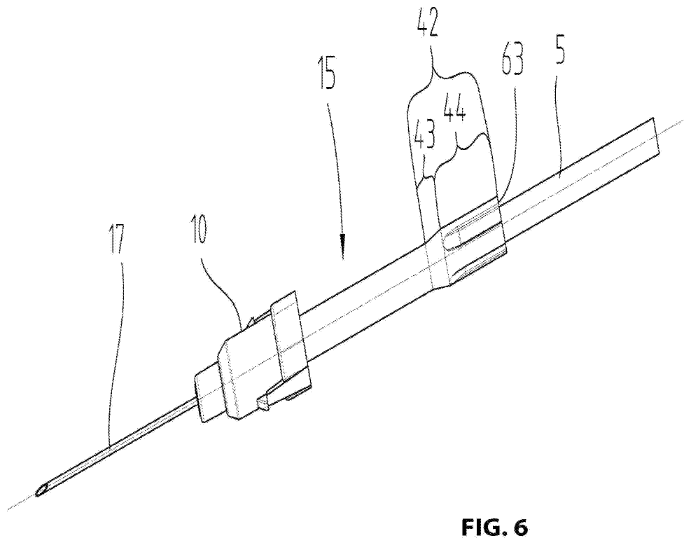

FIG. 6 shows the sliding body 15 including the cannula 17 inserted therein and the tube 5 inserted in the proximal end additionally showing only the first main body part 10. The sliding body 15 includes a socket section 42 that has a greater diameter than the center section and which adjoins in the proximal direction wherein the socket section 42 is formed by a shorter transition section 43 and a substantially cylindrical insertion section 44 adjoining the proximal direction. The insertion section 44 includes an inner cylindrical bore hole that is adapted to an outer diameter of the flexible tube 5 and into which the tube is glued, tightly sealed.

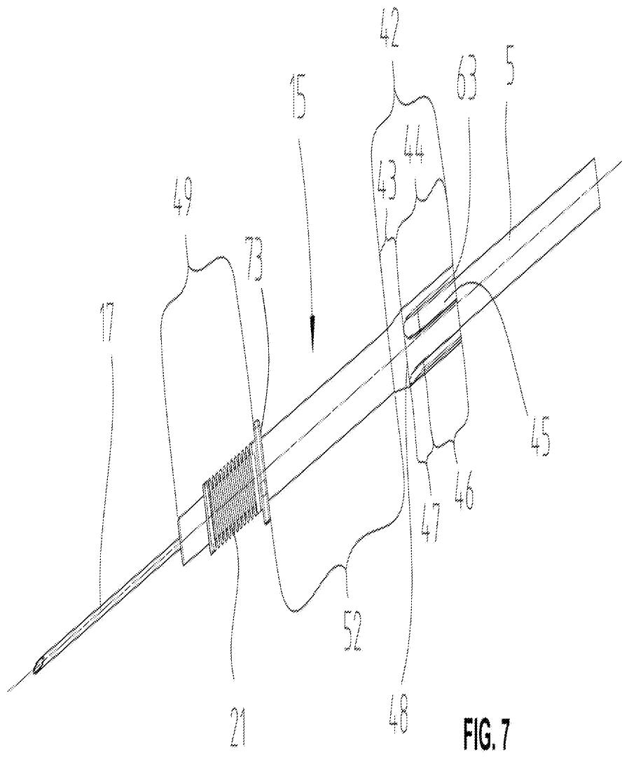

It is evident from FIG. 7 that the socket section 42 includes four stress relief grooves evenly spaced over the circumference of the socket section 42 and running in a direction of the longitudinal axis of the sliding body 15 for blocking tongues of the main body that will be described infra. The stress relief grooves 45 include a longer section 46 where they have a greater depth and a groove base that runs parallel to the longitudinal axis 7 and a shorter distal section 47 where the groove base rises in a ramp to a circumferential border line between the insertion portion 44 and the conical transition section 43. The function of the stress relief grooves in combination with the blocking tongues recited supra will the described infra.

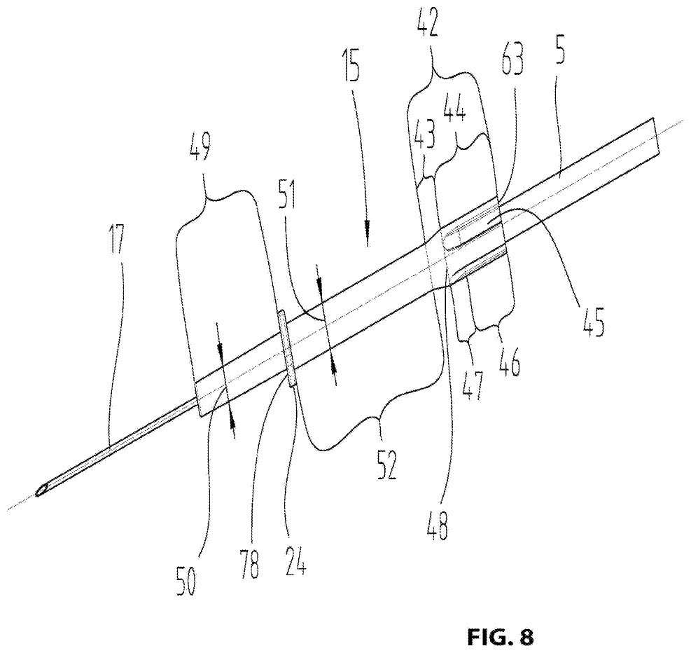

FIG. 7 illustrates the compressed actuation element 21 on a distal end section 49 of the sliding body 15 which is not the case in the otherwise analogous representation of FIG. 8. This indicates that a diameter 50 in the distal end section 49 of the sliding body 15 is greater than a diameter 51 in a center section 52 of the sliding body 15.

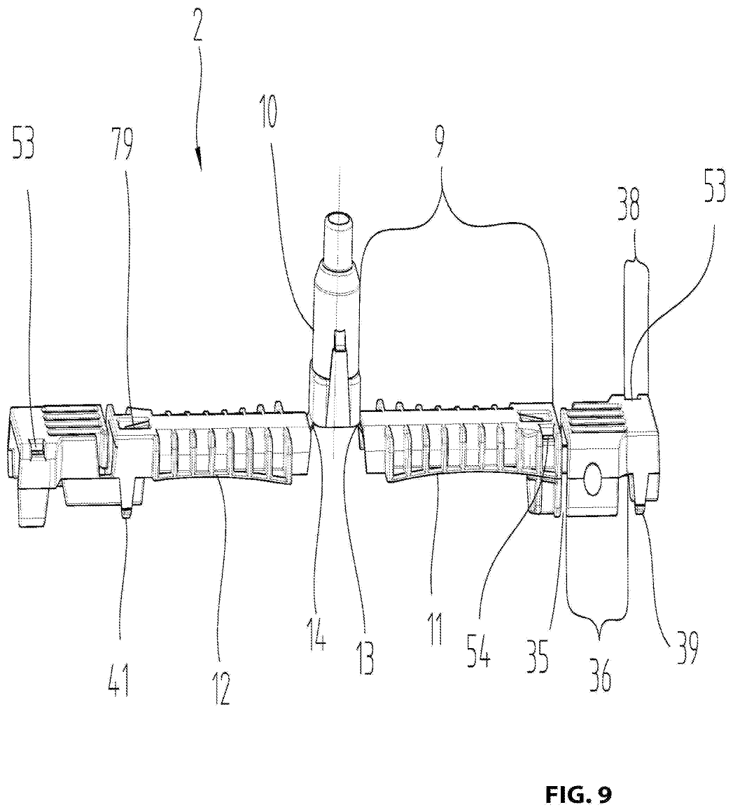

The individual portions of the main body 2 and the mirror symmetrical arrangement of the identically formed main body parts 11 and 12 are evident from FIG. 9 where the main body 2 is shown with the main body parts 11 and 12 laterally folded out from the first main body part 10. In this condition the main body 2 is removed as an injection molded component from the injection molding tool. The grip portion 9, the adjacent notch portion 35, the adjoining trigger portion 36 and the closure portion 38 forming the proximal closure are evident from FIG. 9. A recess 53 is visible on the third main body part 12, shown on the left side of FIG. 9, wherein the snap hook 39 is receivable and lockable in the recess 53 as evident from FIG. 5. Another recess 54 in the grip portion 9 is shown and provides locking reception of the corresponding snap hook 41 at the grip portion 9 of the third main body part 12. Thus, there is a total of two snap hooks 39 and two recesses 53 in the closure portion 38 and two snap hooks 41 and two recesses 54 in the grip portion 9. Each of the two main body parts 11 and 12 includes a respective snap hook 39, 41 and a respective recess 53, 54 in the grip portion 9 as well as in the closure portion 38.

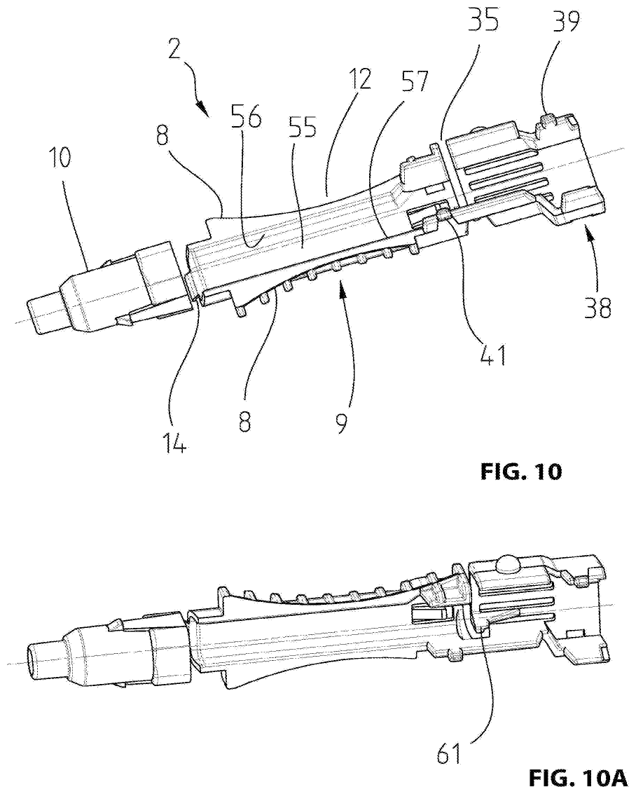

FIG. 10 shows a view into the groove shaped third main body part 12 since the second groove shaped main body 11 part was removed which would otherwise be arranged there above in an assembled condition and on the other hand side also the sliding body 15 and the cannula 17 and the tube 5 are not inserted. It is evident that a base 55 and two opposite half-walls 56, 57 of the main body part 12 are arranged perpendicular to one another which yields a square cross section of the interior cavity when the two main body parts 11, 12 are assembled. The two longitudinal ribs 8 of the main body part 12 arranged on opposite sides are jointly formed by both main body parts 11 and 12 in assembled condition and define a center plane in a contact portion of the two main body parts 11, 12 wherein the center plane runs parallel to the longitudinal axis 7 and also forms a plane of symmetry. FIG. 10 also shows the snap hooks 41 in the grip portion 9 and 39 in the closure portion 38 wherein the snap hooks are arranged on opposite sides in respect to the longitudinal axis 7

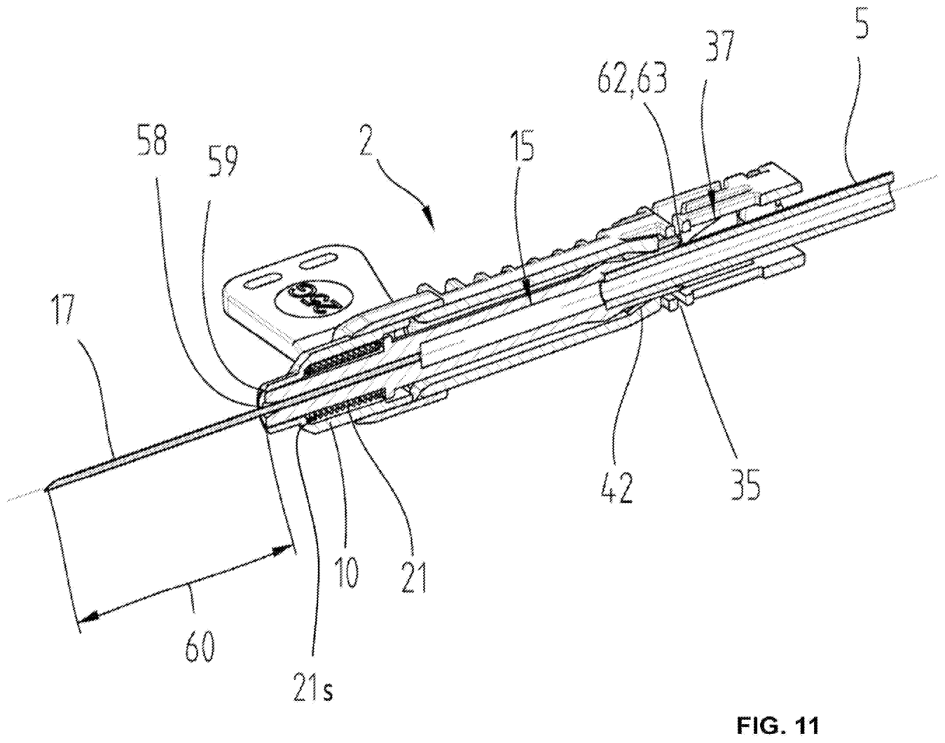

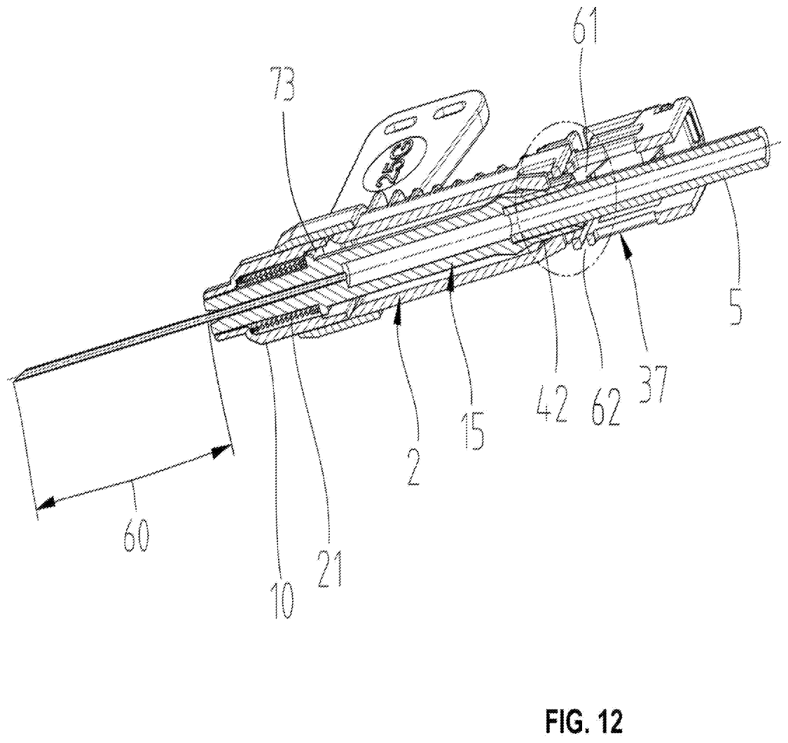

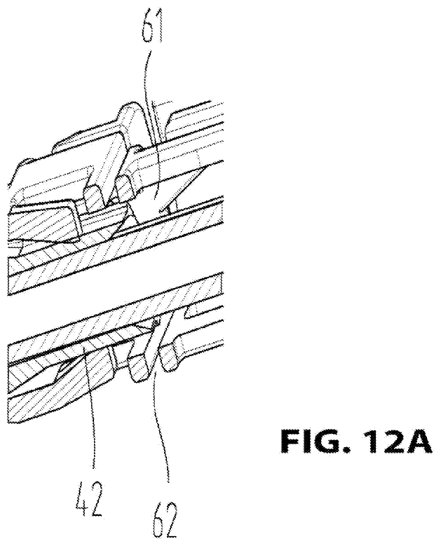

FIGS. 11, 12 and 12A respectively show a longitudinal sectional view of the safety cannula arrangement 1 in an operating condition of the sliding body 15 in different perspectives. A distal face 58 of the sliding body 15 thus eventually terminates flush with a distal face 59 of the first main body part. The cannula 17 protrudes with a free length 60 beyond the distal face 59 of the base body part 10. The actuation element 21 is in a preloaded condition so that the sliding body 15 tends to move relative to the main body 2 in the proximal direction. This movement is prevented by two locking elements 61a, 61b which form part of the trigger mechanism 37 and cooperate with a proximal face 62 of the sliding body 15 in its socket section 42. The face 62 of the sliding body 15 forms a shoulder 63 in combination with the tube 5 that has a smaller diameter which is evident from FIGS. 6 through 8. This shoulder is provided in the operating position 15 viewed in the axial direction in the notched portion 35 at its proximal end that is defined by the locking elements 61a, 61b of the trigger mechanism 37. The sliding body 15 is thus fixed without clearance in the axial direction in the main body 2 in the operating position which is indispensable for performing the puncture correctly.

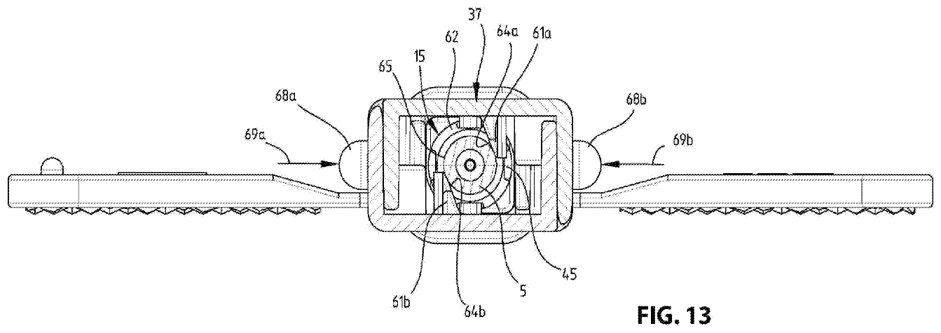

Viewing FIGS. 13 through 16 and 18 together makes the function of the trigger mechanism 37 evident. The proximal face 62 that forms the shoulder 63 in the transition from the tube 5 to the socket section 42 of the sliding body 15 is retained by two diametrically opposed locking elements 61a, 61b, c.f. also FIG. 10A, so that the sliding body 15 remains in the operating position 45. The socket section 42 of the sliding body 15 includes relief grooves which yields a circular contour of the face 62 in a cross section, wherein the circular contour is interrupted by four indentations having a 90-degree offset.

The locking elements 61a, 61b have a triangular or trapezoid shape in an axial viewing direction and contact an outer enveloping surface 65 of the tube 5 with a respective control edge 64a, 64b.

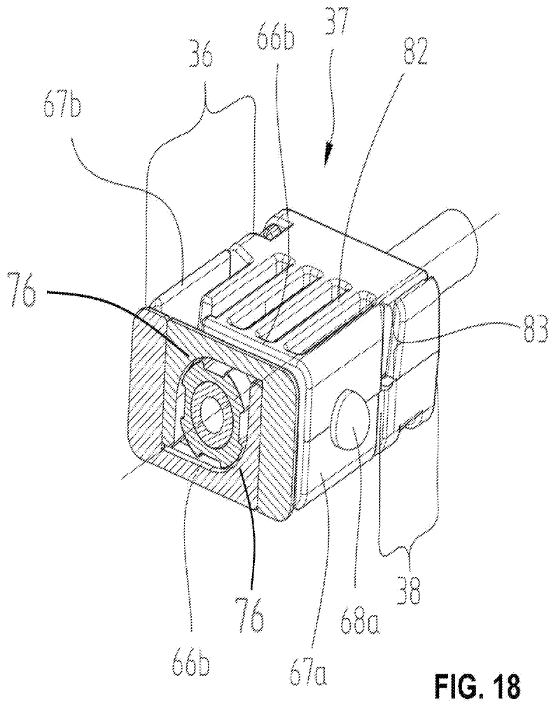

It is evident from FIG. 18 that the locking elements 61a, 61b are coupled respectively by a compression rod 66a, 66b with a trigger member 67a, 67b. The trigger members 67a, 67b are respectably provided with a protrusion 68a, 68b configured as a semi spherical button. The trigger members 67a, 67b form two approximately square surfaces in a side view of the main body 2, the square surfaces having a square protrusion 68a, 68b wherein the compression rods 66a, 66b run at an angle of 90 degrees relative thereto. Thus, the locking elements 61a, 61b run at an angle of approximately 90 degrees relative to the compression rods 66a, 66b so that a C-shape or U-shape is achieved overall. Thus, the trigger members 67a, 67b and the locking elements 61a, 61b respectively form a free arm of the U or C, whereas the compression rods 66a, 66b form a base element of the U or C.

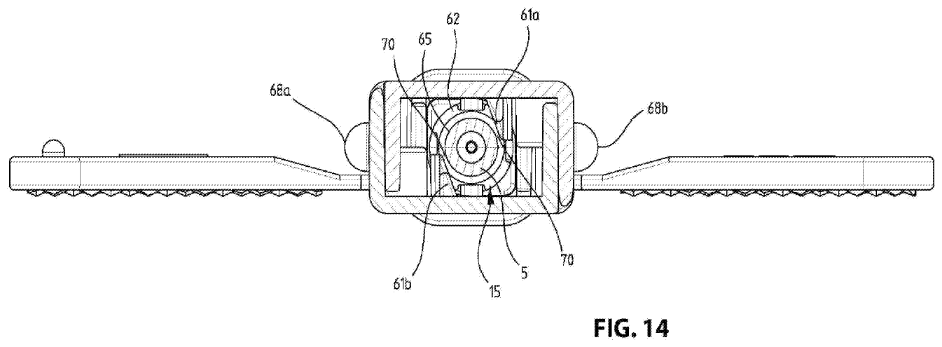

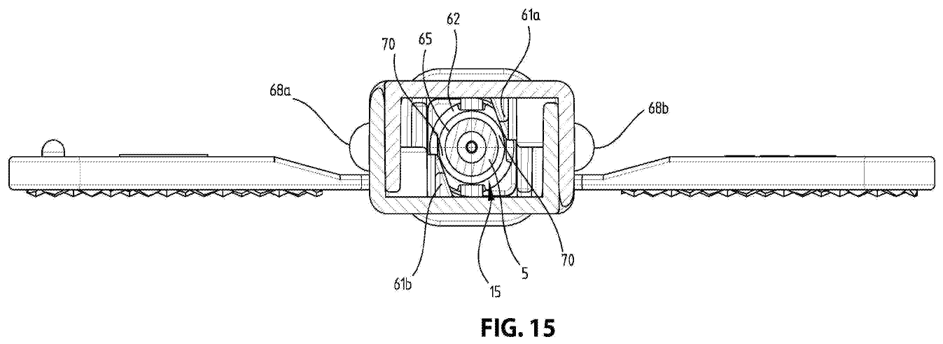

When the sliding body 15 needs to be transferred into the safety position after completing the blood draw or the infusion with the safety cannula arrangement 1 so that the cannula 17 is arranged within the main body 2 in its entirety, the operator of the safety cannula arrangement 1 imparts a pressure force upon the protrusions 68a, 68b of the trigger members 67a, 67a wherein the pressure force is imparted with two fingers of one hand simultaneously and runs in a radial direction according to the arrows 69a, 69b. Starting from the locking position illustrated in FIG. 13 where the locking elements 61a, 61b block the sliding body 15 by an engagement (contact at the face 62) so that the upper locking element 61a in FIG. 13 moves to the right due to pressure upon the left trigger member 68a whereas the lower locking element 61b in FIG. 13 moves to the left due to pressure upon the right trigger member 68b. FIG. 14 shows a condition where a distance 70 has formed between the locking elements 61a, 61b and the outer enveloping surface 65 of the tube 5 due to a displacement of the trigger members 68a, 68b forming a rigid unit and of the locking elements 61a, 61b connected therewith. This distance 70, however, is not large enough yet to release a contact of the face 62 at the locking elements 61a, 61b. Therefore, the cannular carrier 15 remains in the operating position in this position of the trigger mechanism or the trigger members 67a, 67b. FIG. 15 shows another intermediary state where the distances 70 between the locking elements 61a, 61b and the enveloping surface 65 of the tube 5 are larger but still cause an overlap between the locking elements 61a, 61b and the face 62 of the sliding body 15 and thus a blocking of the sliding body 15.

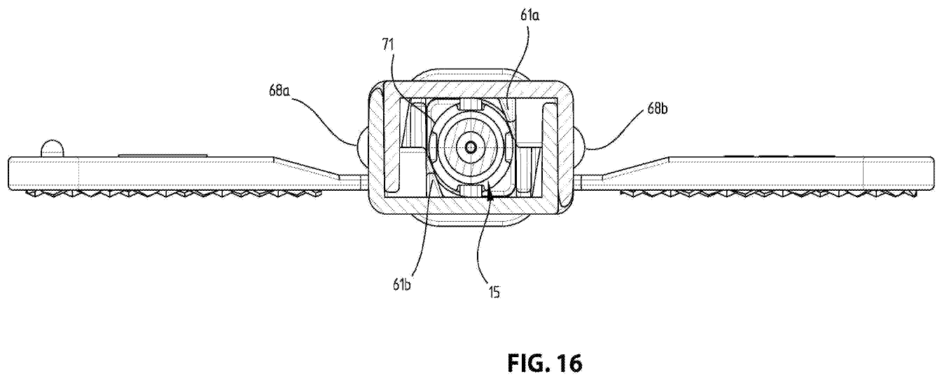

When the trigger members 67a, 67b with their associated knob shaped protrusions 68a, 68b are in the position shown in FIG. 16 the associated locking elements 61a, 62b are displaced outward enough so that the cross section of the sliding body 15 defined by a circular enveloping line 71 is released in its entirety in the portion of the socket section 42. Now, the locking elements 61a, 61b are out of engagement with the sliding body 15 so that the sliding body 15 can move in the proximal direction driven by the actuation element 21 which causes a pull back of the cannula 17 into the interior of the main body 2.

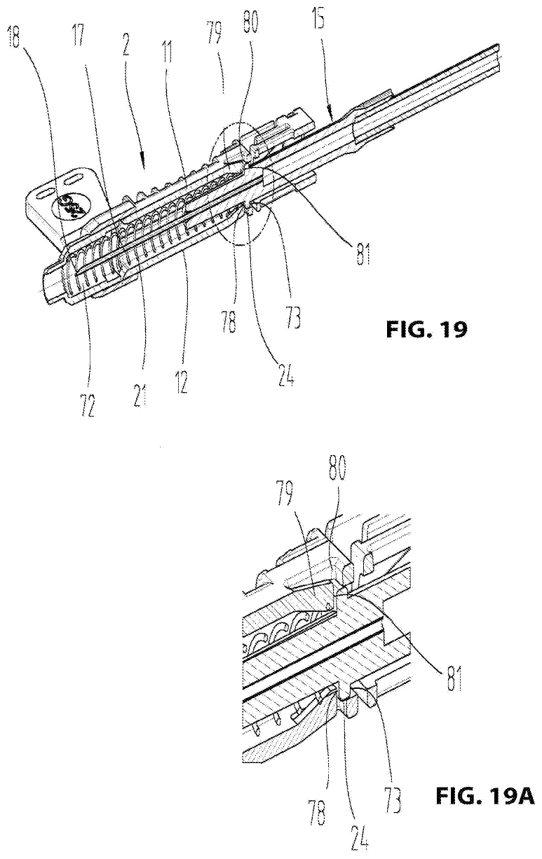

Accordingly, FIGS. 19 and 19A show the sliding body 15 in the safety position where the cannula 17 including the tip 18 is arranged within an interior cavity 72 of the main body 2. The actuation element 21 configured as the coil spring is now provided in a condition where it has less tension than in the operating position of the sliding body 15.

In order to prevent an exit of the entire sliding body 15 and thus also of the cannula 17 itself at the proximal end of the main body 2 the safety position of the sliding body 15 is defined by a stop surface 73 arranged at the sliding body 15. The stop surface 73 arranged at the sliding body 15 thus cooperates with a stop surface 74 of the main body 2 that is formed at the proximal end of the main body 2, thus at the proximal end of the closure portion 38 which is formed jointly by the second main body part 11 and the third main body part 12.

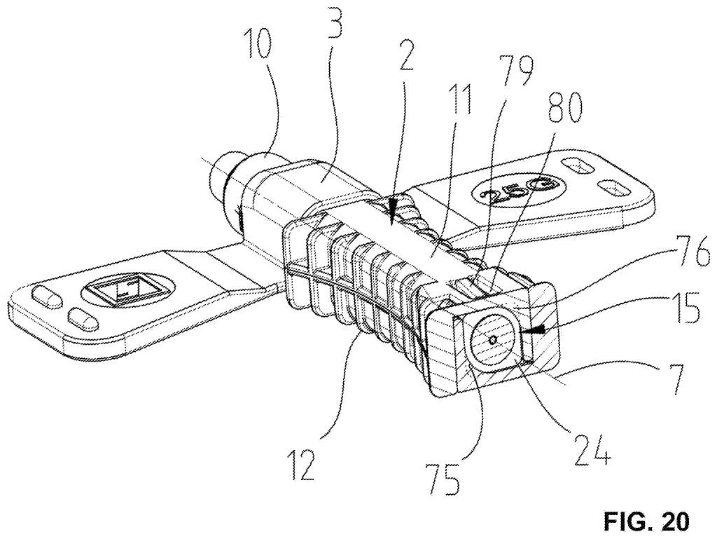

It is evident from the sectional view of FIG. 20 that the second main body part 11 and the third main body part 12 are U-shaped in cross section wherein the two U-shaped cross sections of the walls are nested into one another in a direction perpendicular to the plane of symmetry of the main body 2, this means in a direction perpendicular to the two film hinges, this means of the pivoting motion during the joining process and thus define an essentially square free cross section in the interior cavity 72 of the main body 2. The cross-section shape in the portion of the stop surfaces 74 of the main body parts 11 and 12, however is not exactly square but two corner portions are filled and defined towards the longitudinal axis 7 of the main body 2 respectively by an arcuate line 75 which forms a quarter circle in the respective corner of the U or C. FIG. 20 shows the edges of an imaginary square where the stop segments 76 defined by the arcuate line 75 are introduced into the corners in dashed lines for the purpose of clarity.

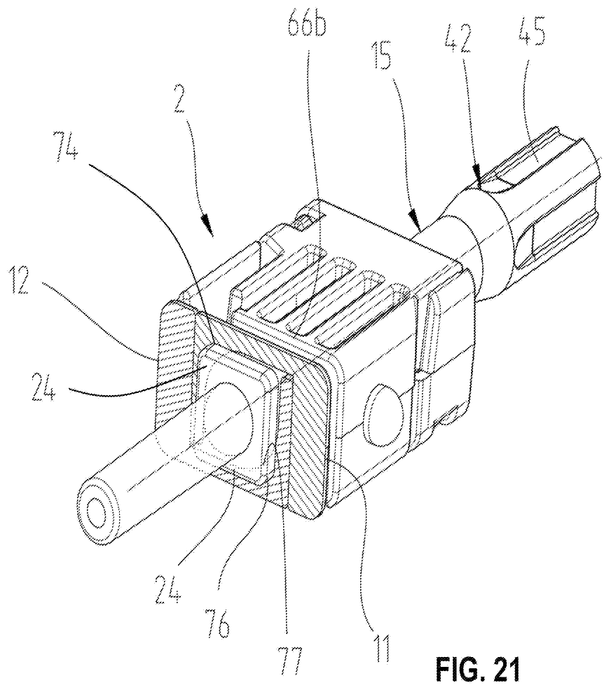

As evident from FIG. 20, the radius of a circle that runs through the slightly rounded corners of the square flange 24, is greater viewed in the direction of the longitudinal axis 7 than a radius of a circle which is formed by a continuation of the two arcuate lines 75. Therefore, the square flange 24 cannot pass through the free cross section in the area of the stop surfaces 74 that are formed at the stop segments 76, so that a displacement of the sliding body 15 stops at this location. This is also clearly evident from the sectional view according to FIG. 21. The square flange 24 contacts the stop surfaces 76 of the main body parts 11, 12 in the portion of the rounded sections 77. The cannula carrier and sliding body 15 is in the safety position in the condition illustrated in FIG. 21 where the socket section 42 has exited from the main body 2 at a proximal end of the sliding body 15.

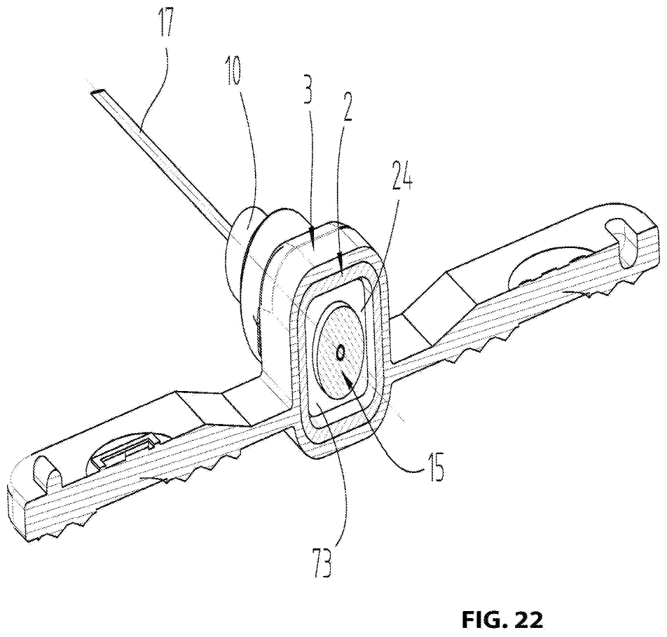

It is evident from FIG. 22 that the interior cavity 72 in the cuboid section 27 of the overall sleeve shaped first main body part 10 is square with rounded corners. The cross section of the square flange 24 is adapted to this cross-section shape so that a smooth low resistance movement of the sliding body 15 is facilitated in the axial direction under an impact of the force of the actuation element 21 and a rotation of the sliding body 15 about the longitudinal axis 7 is reliably prevented. Thus, the flange 24 performs the function of a rotation blocking body of the sliding body 15 and the corresponding cuboid section 27 of the first main body part 10 forms a rotation blocking body of the main body 2. While the proximal contact surface 73 is arranged at the square flange 24 of the sliding body 15 defining the pull back movement of the sliding body 15 the square flange 24 has a double function since its additional stop surface 78 that is not shown in FIG. 22, but shown in FIGS. 7 and 8 supports the actuation element 21.

In addition to limiting the pull back movement of the sliding body 15 in the proximal direction it is also important to prevent that the sliding body 15, after having moved into the safety position, is moved back in the distal direction intentionally or unintentionally so that the cannula 17 is exposed again and the safety cannula arrangement 1 may be used again which shall be avoided and prevented under all circumstances. The blocking tongues 79 recited supra serve this purpose wherein the blocking tongues are configured as spring elastic elements of the main body 2 and respectively arranged within a window 80 enveloping the blocking tongues 79 on three sides. This configuration is clearly evident from FIGS. 19 and 19A, where the sliding body 15 is in the safety position. A respective face 81 of the blocking tongues 79 contacts the distally oriented contact surface 78 of the square flange 24 so that the square flange 24 performs three functions: end stop for the pull back movement, support of the actuation element 21 and stop surface for the blocking tongues 79. Since the blocking tongues 79 are elastically connected with the respective main body part 11, 12 the blocking tongues 79 move elastically radially outward when the sliding body 15 passes through during the pull back movement and move back radially inward after the passage of the square flange 24 due to the preload of the blocking tongues 79 and perform the blocking in cooperation with the flange 24. Since the diameter of the sliding body 15 in the distal end section 49 is smaller than in the center portion 52 a sufficiently large overlap of the blocking tongues 79 with the protrusion of the flange 24 beyond the adjacent cylindrical surface is provided viewed in radial direction.

As evident from FIG. 9 the blocking tongues 79 are formed during injection molding of the main body parts 11, 12 so that they are inclined relative to the longitudinal axis 7 of the main body 2 or the sliding body 15 starting from a connection cross section with the associated main body part 11, 12 and moving towards their free ends. In order to maintain this preload radially inward reliably even during a long storage time of the safety cannula arrangement 1 relief grooves 45 are formed in the socket section 42 of the sliding body 15 as described supra. The relief grooves facilitate an orientation of the blocking tongues 79 that is inclined relative to the longitudinal axis 7 during storage in the operating condition of the sliding body exactly in the shape of the relief tongues that is subsequently required in the safety position of the sliding body 15 to block another deployment movement of the sliding body 15. This way material fatigue and a loss of the radially inward oriented preload is prevented compared to a configuration of the socket section 42 without the relief grooves 45 which could otherwise lead to a failure of the blocking tongues 79 so that a redeployment of the cannula 17 would not be prevented reliably.

The following is appreciated regarding the configuration of the trigger mechanism 37 with reference to FIG. 18, as well as FIGS. 10, 10A and 4.

As stated supra, the main body 2 is substantially cuboid in the trigger portion 36, wherein a respective trigger member 67a, 67b and the associated compression rod 66a, 66b in combination with link elements configured as bar 82 jointly form a L-shaped wall arrangement, wherein the three link elements are connected by the respective compression rod 66a, 66b. Due to the small cross sections of the bars 82, they can be deformed easily by pressure upon the trigger members 67a relative to the proximal closure portion 38. When pressure is imparted upon the trigger members 67a, 67b, a connection cross section 83 arranged between the trigger members 67a, 67b and the closure portion 38 functions as an additional pivot joint. A trigger surface respectively formed by the trigger member 67a, 67b runs at an angle of 90 degrees relative to a plane in which the bars 82 and the respective compression rod 66a, 66b connecting the bars 82 are arranged. The respective locking elements 61a, 61b extend at an angle of 90 degrees relative the plane of the bars 82, and the respectively associated compression rod 66a, 66b. Overall, a C- or U-arrangement is achieved in cross section.

### REFERENCE NUMERALS AND DESIGNATIONS

1 safety cannula arrangement
2 main body
3 lobe module
4 cannula protector
5 tube
6 transversal rib
7 longitudinal rib
8 longitudinal rib
9 grip portion
10 first main body part
11 second main body part
12 third main body part
13 film hinge
14 film hinge
   15 sliding body
   15m enveloping surface
   16 distal end section
   17 cannula
   18 tip
   19 proximal end section
   20 distal end section
   21 actuation element
   21s support surface
   22 distal end
   23 proximal end
   24 flange
   25 interlocking lug
   26 rotation symmetrical section
   27 cuboid section
   28 tip section
   29 transition section
   30 connection part
   31 gripping lobe
   32 length
   33 length
   34 overlapping portion
   34d section
   34p section
   35 notched portion
   36 trigger portion
   37 trigger mechanism
   38 closure portion
   39 snap hook
   40 connection bar
   41 snap hook
   42 socket section
   43 transition section
   44 plug in section
   45 stress relief groove
   46 section
   47 section
   48 boundary line
   49 end section
   50 diameter
   51 diameter
   52 center portion
   53 recess
   54 recess
   55 base
   56 semi-wall
   57 semi-wall
   58 face
   59 face
   60 free length
   61a, 61b locking element
   62 proximal face
   63 shoulder
   64a, 64b control edge
   65 enveloping surface
   66a, 66b compression rod
   67a, 67b trigger member
   68a, 68b protrusion
   69a, 69b arrow
   70 distance
   71 circumferential line
   72 inner cavity
   73 stop surface
   74 stop surface
   75 arcuate line
   76 stop element
   77 section
   78 stop surface
   79 blocking tongue
   80 window
   81 face
   82 bar
   83 connection cross section
   84 rotation blocking device

What is claimed is:

1. A safety cannula assembly, comprising:
a cannula configured to puncture human or animal tissue, wherein a distal end section of the cannula is provided with a tip;
a sliding body including a distal end section including the cannula and a proximal end section including a flexible tube, wherein a flow connection between the tip of the cannula and a proximal end of the flexible tube runs through the sliding body;
a main body in which the sliding body is displaceable from an operating position where the tip of the cannula is arranged outside of the main body into a safety position where the tip of the cannula is arranged inside the main body, wherein the main body is assembled from at least three main body parts, including
a first main body part configured sleeve shaped and enveloping a distal end section of the sliding body in the operating position of the sliding body,
a second main body part adjoining the first main body part in the proximal direction and connected with the first main body part, wherein the second main body part has a U-shaped, L-shaped, C-shaped, or lid-shaped cross section,
a third main body part joinable with the second main body part to form a hollow body that is open at two opposite faces, wherein the third main body part is configured lid-shaped, C-shaped, L-shaped, or U-shaped and connected by a film hinge with the second main body part or the first main body part;
an actuation element arranged between the main body and the sliding body and configured to displace the sliding body from the operating position to the safety position; and
a trigger mechanism arranged at the main body and configured to trigger a displacement of the sliding body from the operating position to the safety position, wherein the trigger mechanism includes at least one trigger member loadable with a radially oriented compressive force or a torque by a person using the safety cannula assembly, and at least one locking element displaceable by loading with the pressure force or the torque from a locking position where the locking element engages the sliding body and locks the sliding body in the operating position to a trigger position, where the locking element does not engage the sliding body so that the sliding body moves into the safety position,
wherein the actuation element is supported at a support surface of the first main body part with a distal end of the actuation element.

2. The safety cannula assembly according to claim 1, wherein the main body includes a respective gripping lobe at two opposite sides.

3. The safety cannula assembly according to claim 2, wherein the actuation element is supported with a distal end at a shoulder formed in an interior cavity of the first main body part or at a face wall forming a distal termination of the first main body part.

4. The safety cannula assembly according to one of the claim 3, wherein the gripping lobes are connected at a sleeve shaped connector that is slid onto the main body, wherein an overlapping portion between the connector and the main body envelops at least an axial section of the first main body part as well as at least a respective axial section of the second main body part and the third main body part.

5. The safety cannula assembly according to claim 4, wherein the first main body part and the second main body part are fixed at one another in a rigid manner and integrally configured in one piece.

6. The safety cannula assembly according to claim 4,
wherein the first main body part and the second main body part are connected with one another by the film hinge, and
wherein a hinge axis of the film hinge is oriented perpendicular to a longitudinal axis of the sliding body and arranged at a distance from the longitudinal axis of the sliding body.

7. The safety cannula assembly according to claim 6,
wherein the third main body part and the second main body part are connected with one another by a film hinge, and
wherein a hinge axis of the film hinge is oriented parallel or perpendicular to the longitudinal axis of the sliding body.

8. The safety cannula assembly according to claim 6,
wherein the third main body part and the first main body part are connected by a film hinge, and
wherein a hinge axis of the film hinge is oriented perpendicular to and at a distance from the longitudinal axis of the sliding body.

9. The safety cannula assembly according to claim 8, wherein the two film hinges have an identical distance from the tip of the cannula viewed in the axial direction.

10. The safety cannula assembly according to claim 9, wherein the two film hinges respectively have an identical distance from the longitudinal axis of the sliding body.

11. The safety cannula assembly according to one of the claim 10, wherein the first main body part
is rotation symmetrical in a distal section and/or
is cuboid in a proximal section,
wherein a respective film hinge is arranged at opposite proximal edges of the cuboid proximal section.

12. The safety cannula assembly according to claim 11,
wherein the rotation symmetrical distal section of the first main body part has a cylindrical tip section and a cylindrical transition section configured to receive the actuation element that is compressed in the operating position,
wherein a removable tubular cannula protector is slid onto the tip section.

13. The safety cannula assembly according to claim 12, wherein the second main body part and the third main body part in assembled condition jointly form
a gripping portion adjoining the film hinges in the proximal direction, wherein the gripping portion is configured with ribs or a plurality of protrusions on its outer enveloping surface to improve gripping, and/or
a trigger portion adjoining the gripping portion in the proximal direction and including the trigger mechanism, and/or
a closure portion formed at a proximal end of the main body, wherein the second main body part and the third main body part are connected by snap hooks and/or interlocking lugs in the closure portion by positive form locking.

14. The safety cannula assembly according to one of the claim 13, wherein the first main body part forms a distal end of the main body.
